(19) United States
(12) Patent Application Publication
(10) Pub. No.: US 2026/0034291 A1
(43) Pub. Date: Feb. 5, 2026

(54) FLUID TRANSFER DEVICE

(71) Applicant: SPOKE MEDICAL LIMITED, Leeds (GB)

(72) Inventors: Patrick Robert Axon, Cambridge (GB); Neil Patrick Donnelly, Cambridge (GB)

(21) Appl. No.: 19/137,350

(22) PCT Filed: Dec. 13, 2023
(86) PCT No.: PCT/GB2023/053209

§ 371 Date: Jun. 10, 2025

(30) Foreign Application Priority Data
Dec. 22, 2022 (GB) 2219508.5

(51) Int. Cl. A61M 1/00

(52) U.S. Cl. CPC A61M 1/7411; A61M 1/842; A61M 2205/3334; A61M 2205/586

(57) ABSTRACT

The present application describes a device (100) for transferring a fluid from a surgical site, comprising an elongate body portion (102) defining a proximal end region (106) of the device connectable to a source of negative pressure, an elongate neck portion (104) defining a distal end region (108) of the device locatable at a surgical site, a through bore extending from the distal end region to the proximal end region, and a control member (116) for selectively controlling a negative pressure at the distal end region of the device, wherein the control member comprises a button portion actuatable by a user to selectively move a valve portion of the control member across the through bore from an open position towards a closed position to at least partially restrict a fluid flowing along the through bore. A system including said device (100) and a flexible tube (200) and a method of controlling a negative pressure at the distal end region of the device are also described.

The present invention relates to a device for transferring solids and/or fluids from a location, and in particular, but not exclusively, to a suction device for use in, for example, the medical industry during a surgical procedure.

A medical suction device is often used by a surgeon or an assistant to remove fluid from a surgical site during a surgical or investigative procedure, such as an abdominal procedure or an ear, nose and throat (ENT) procedure. A conventional ENT suction device typically comprises a tube made from stainless-steel and having an outer diameter of around 1-5 mm. The tube typically extends from a plastic attachment portion for coupling the tube to a pump to create a negative pressure at a distal end of the tube. The attachment portion connects by way of an interference fit or threaded connection to a length of flexible tubing coupled to the pump. The attachment portion may be configured to allow the surgeon or assistant to hold the device and manoeuvre the suction tube in use.

However, conventional medical suction devices have no or very limited function to allow a user to efficiently, accurately and selectively control the negative suction pressure at the distal end of the tube locatable at a surgical site. Uncontrolled suction forces can cause trauma to delicate organs leading to unintended injury and patient morbidity. Furthermore, manoeuvring the suction device during a surgical procedure often causes the flexible tubing between the device and the vacuum pump to twist which in turn applies a reaction torque to the suction device itself. This reaction torque can undesirably restrict the user when manoeuvring the suction device and can result in the suction tube unintentionally rotating which could cause discomfort to the patient. In addition, conventional medical suction devices are prone to blocking, particularly at the tip and around connections, e.g. between the device and the flexible tubing, when for example wax, clotted blood, bone fragments, or the like, block the tube at least partially, and often completely, which results in a significant reduction in performance of the suction device and in turn undesirable downtime of the surgical procedure, increased waste and cost.

Conventional medical suction devices are also typically noisy to use in view of turbulence caused within the device and the constant vacuum being applied at the distal end of the suction tube when the device is not being used. This can be frustrating and distracting to a surgeon and when the device is being used close to the ear the relatively high noise levels can cause acoustic trauma (hearing loss, hyperacusis and tinnitus) to the patient. Furthermore, conventional medical suction devices are uncomfortable to use for relatively long periods of time and/or during surgical procedures which require high levels of accuracy from the surgeon and/or their assistant.

A medical suction device is described in WO2021/140316. Whilst this device is a significant improvement upon previously available medical suction devices and addresses many of the abovementioned problems, the applicant has further improved the device as described herein.

It is an aim of certain embodiments of the present invention to provide a medical device for accurately and efficiently transferring a solid and/or fluid from a surgical site.

It is an aim of certain embodiments of the present invention to provide a medical suction device for use by a surgeon which is comfortable to use for relatively long periods of time.

It is an aim of certain embodiments of the present invention to provide a medical suction device which allows a surgeon to accurately and efficiently control the amount of suction applied by the device at a surgical site and also noise intensity.

It is an aim of certain embodiments of the present invention to provide a medical suction device which is easy to connect to a vacuum source, such as a pump, and which is not adversely affected by the flexible tubing coupling the device to the vacuum source.

It is an aim of certain embodiments of the present invention to provide a medical suction device which can be used with a selected one of a plurality of differently configured suction tubes based on the surgical procedure and/or the surgical site.

It is an aim of certain embodiments of the present invention to provide a medical suction device which is less prone to blocking and which is significantly quieter than conventional medical suction devices, particularly when not being used.

It is an aim of certain embodiments of the present invention to provide a medical suction device configured to avoid uncontrolled and excessive negative pressure being applied at a surgical site, such as in the ear, which could otherwise cause trauma to delicate organs leading to unintended injury and patient morbidity.

It is an aim of certain embodiments of the present invention to provide a medical suction device configured to allow wax to be gently removed from an ear canal at substantially low noise levels to reduce/eliminate the risk of acoustic shock.

It is an aim of certain embodiments of the present invention to provide a medical suction device configured to allow a user to implant relatively small prostheses, such as ear grommets, with the device.

According to a first aspect of the present invention there is provided a device for transferring a fluid from a surgical site, comprising:
an elongate body portion defining a proximal end region of the device connectable to a source of negative pressure;
an elongate neck portion defining a distal end region of the device locatable at a surgical site;
a through bore extending from the distal end region to the proximal end region; and
a control member for selectively controlling a negative pressure at the distal end region of the device, wherein the control member comprises a button portion actuatable by a user to selectively move a valve portion of the control member across the through bore from an open position towards a closed position to at least partially restrict a fluid flowing along the through bore.

Optionally, the control member is slidably mounted in a cavity of the body portion such that the valve portion is moveable towards the closed position responsive to the button portion being pressed by the user.

Optionally, the valve portion of the control member is slidably mounted in a guide bore extending substantially perpendicularly into the through bore.

Optionally, the guide bore is defined by a hollow boss extending upwardly from a base of the cavity.

Optionally, the control member comprises a vent bore extending from an upper surface of the button portion towards a closed base of the valve portion, and a vent hole disposed in a distal side of the valve portion such that the vent hole is closed off by the guide bore when the control member is in the open position and the vent hole is located in fluid communication with a distal bore region of the through bore when the control member is in the closed position.

Optionally, the lower region of the valve portion comprises a curved surface for slidably engaging a correspondingly curved seat surface of the through bore which is a continuation of the guide bore and defined at an intersection of a distal bore region and a proximal bore region of the through bore.

Optionally, a lower region of the valve portion comprises a substantially flat base surface for engagement with a correspondingly flat base seat surface of the proximal bore region when the valve portion is in the closed position, and wherein the lower region of the valve portion further comprises substantially tapered and flat side surfaces for engagement with correspondingly tapered and flat side seat surfaces of the proximal bore region when the valve portion is in the closed position.

Optionally, the valve portion of the control member is biased towards the open position by a biasing element, and the button portion of the control member comprises a first abutment surface for engagement with a first stop surface of the body portion of the device when the valve portion is in the open position and to limit travel thereof.

Optionally, the first abutment surface is provided by a first flange region extending proximally from a lower region of the button portion, and the first stop surface is provided by a lip region extending distally from an upper edge region of the cavity.

Optionally, the button portion comprises a second abutment surface for engagement with a second stop surface of the body portion when the valve portion is in the closed position and to limit travel thereof.

Optionally, the second abutment surface is provided by a second flange region extending proximally from an upper region of the button portion, and the second stop surface is provided by the lip region.

Optionally, the button portion comprises a third abutment surface for engagement with the second stop surface to hold the valve portion in a locked position.

Optionally, the third abutment surface is provided by the second flange region and the second stop surface is provided by the lip region.

Optionally, the second flange region and/or the lip region is substantially resilient to allow the second flange region to be urged past the lip region and engage thereunder when the control member is moved from the closed position to the locked position.

Optionally, the button portion is tiltable towards the proximal end region of the device to locate the second flange region under the lip region and/or is tiltable towards the distal end region of the device to release the second flange region from under the lip region.

Optionally, a minimal amount of fluid is allowed to flow along the through bore and past the valve portion when the same in the closed position, and wherein substantially no fluid is allowed to flow along the through bore and past the valve portion when the same is in the locked position.

Optionally, the curved surface of the valve portion is urged against the curved seat surface of the through bore when the valve portion is in the locked position.

Optionally, an inner surface of the through bore tapers outwardly in a distal to proximal direction.

According to a second aspect of the present invention there is provided a system for transferring a fluid from a surgical site, comprising:
a device according to the first aspect of the present invention;
at least one suction tube mountable on the distal end region of the device; and
optionally further comprising a flexible tube connectable to the proximal end region and rotatable with respect to the device.

According to a third aspect of the present invention there is provided use of a device according to the first aspect of the present invention or a system according to the second aspect of the present invention for transferring a fluid or solid from a target site.

According to a fourth aspect of the present invention there is provided a method of selectively controlling a negative pressure at a distal end region of a suction device, comprising:
actuating a button portion of a control member of the suction device to move a valve portion of the control member across a through bore of the device from an open position to a closed position to substantially restrict a fluid flowing along the through bore from the distal end region and past the valve portion to thereby substantially reduce but not stop the negative pressure at the distal end region of the device.

Optionally, actuating comprises pressing the button portion.

Optionally, the method comprises picking up an object at the distal end region of the device when the valve portion is in the closed position.

Optionally, the object comprises a medical implant.

Optionally, the method comprises moving the control member from the closed position to a locked position wherein at least one seal surface of the valve portion is urged against a respective seat surface of the through bore to thereby prevent fluid flow past the valve portion.

Optionally, the method comprises at least partially removing a user's finger or thumb from the button portion to at least partially uncover an opening in the button portion to allow a negative pressure in a distal bore region of the through bore and upstream of the valve portion to release to atmosphere via a vent of the control member in fluid communication with the distal bore region when the valve portion is in the locked position.

### DESCRIPTION OF THE DRAWINGS

Certain embodiments of the present invention will now be described with reference to the accompanying drawings in which:

FIG. 1 illustrates an isometric view of a suction device according to certain embodiments of the present invention;

FIG. 2 illustrates a cross section through the device of FIG. 1;

FIGS. 3a to 3c illustrate sectional views through the device when a control member thereof is in an open position;

FIGS. 4a to 4c illustrate sectional views through the device when the control member thereof is in a closed position;

FIG. 5 illustrates a sectional view of a suction tube attached to the distal end of the device;

FIG. 6a illustrates a flexible tube connected to the proximal end of the device;

FIG. 6b illustrates a sectional view through the proximal end of the device and a distal end of the tube connected thereto; and

FIGS. 6c and 6d illustrate the flexible tube attachable to the proximal end of the device.

### DETAILED DESCRIPTION

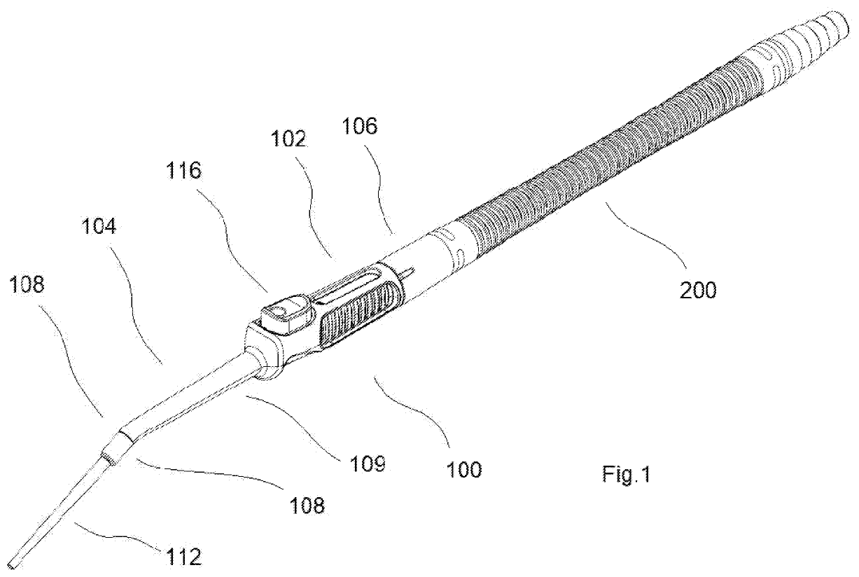

As illustrated in FIG. 1, a suction device 100 according to certain embodiments of the present invention includes an elongate body portion 102 and an elongate neck portion 104 extending therefrom. The body portion 102 defines a proximal end region 106 of the device for attaching a flexible tube 200 thereto which is coupled in use to a source of negative pressure, such as a vacuum pump, and the neck portion 104 defines a distal end region 108 of the device for mounting a suction tube 112 thereto. The device 100 further includes a control member 116 to allow a user to selectively stop, start and control the level of suction provided at the distal end of the suction tube 112 in use, as described further below.

A central axis of the distal end region 108 of the neck portion 104 of the device is angled with respect to a central axis of a proximal region 109 of the neck portion 104. The angle is around 25 degrees, but may aptly be from 0 degrees (i.e. coaxial with the proximal region 109) to around 45 degrees, or any angle suitable for a particular surgical application. A central axis of the proximal region 109 of the neck portion 104 is substantially coaxial with an axis of the body portion 102. The central axes of the distal and proximal end regions of the neck portion may be substantially linear or they may be curved to provide a curved continuous axis extending through a substantially curved neck portion. Providing a neck portion 104 having an angled distal end region 108 desirably allows a selected one of a plurality of suction tubes/nozzles to be mounted to the device. As illustrated, the suction tube 112 may be substantially straight. Whilst curved suction tubes having a bend therein may be used with the device, straight suction tubes 112, i.e. tubes without a bend, are relatively non-complex and inexpensive to manufacture and are also easier and less costly to sterilise or recycle than angled/bent suction tubes. Furthermore, alternatively configured attachments may be coupled to the distal end of the device, such as a straight, curved or right-angled needle, or an elongate probing device that neither sucks, blows or dispenses, for example. Such attachments may be selectively interchangeable by the user for different applications during a surgical procedure. Whilst the outer surface of the distal end region of the neck portion as illustrated is substantially circular in cross section to couple with a correspondingly shaped attachment portion of a suction tube or the like, the distal end region may alternatively be substantially square, elliptical, hexagonal or the like. The tip region of the suction tube 112 may be substantially flexible to absorb impacts with relatively hard tissue, such as inside the ear, and minimise any discomfort to a patient. The tip opening may be bevelled to avoid any sharp edges and to guide and encourage fluids and solids into the suction tube in use.

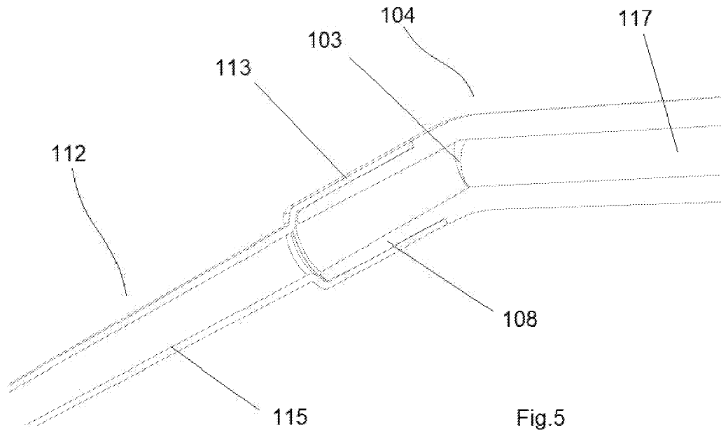

As illustrated in FIG. 5, the suction tube/nozzle 112 mountable to the device according to certain embodiments of the present invention may be a one-piece stainless steel or plastics component having an attachment portion 113 configured to mount to the distal end region 108 of the neck portion 104 of the device by way of a simple inference fit, for example. An inner surface 115 through the suction tube 112 tapers outwardly along its length from its tip, but alternatively the inner surface may be parallel sided. The distal end region 108 of the neck portion 104 of the device and the attachment portion 113 of the suction tube/s 112 are configured to provide a sealed connection therebetween and a substantially continuous and smooth inner wall surface to at least minimise any steps, cervices, etc. which could cause turbulence effects, such as noise, and to prevent blocking in use. Aptly, the suction tube is formed from a plastics/polymer material, such as Polyoxymethylene (POM) in view of its relatively low coefficient of friction allowing the suction tube to be efficiently mounted on and removed from the distal end of the device and also to at least minimise, if not prevent, blocking in use.

An optional cap or cover (not shown) having a closed distal end may be mountable over the suction tube to protect the same and prevent ingress of dirt or the like into the tube and device when not in use. Alternatively, the distal end of the cover may be open to aid manufacturing, for example. Aptly, the cover may provide a visual and/or tactile indication that the suction tube has been used previously on a different patient and is therefore no longer clean for use. For example, the cover may be coloured and/or may be coupled to a portion of the suction tube by a frangible region which is compromised when the cover is removed from the tube. Once the cover has been removed by a first user, it is clear to a second user that the suction tube has previously been used.

The body portion 102 and the neck portion 104 are substantially circular in cross section and are integrally formed by suitable means, such as injection moulding or the like. Aptly, the body portion 102 and the neck portion 104 are formed from a plastics material, such as a polycarbonate. The body portion 102 has a maximum outer diameter which is greater than a maximum outer diameter of the neck portion 104. The outer surface of the body portion 102 tapers inwardly towards the proximal end region 106 and the outer surface of the neck portion 104 tapers inwardly towards the distal end region 108. The body portion 102 is sized and shaped to ergonomically fit in a user's hand, whilst allowing the thumb or a finger to comfortably engage and selectively actuate the control member 116. The body portion 102 may be formed from a material, such as a plastic or rubber, having a relatively high coefficient of friction compared to the neck portion 104, or may include one or more such portions/surfaces, to provide grip to a user when holding the device. The control member 116 is aptly a one-piece component formed by injection moulding or the like. Aptly, the control member 116 is formed from a plastics/polymer material, such as Polyoxymethylene (POM) or the like and may include an upper surface texture and/or material to provide grip when a user's thumb or finger is engaged therewith.

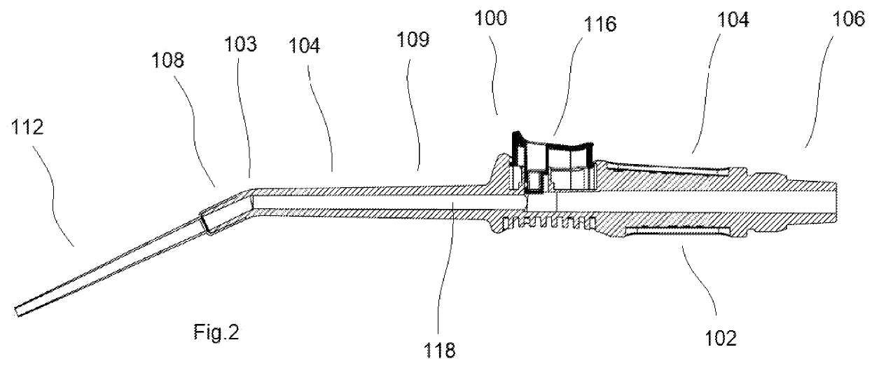

As illustrated in FIG. 2, the device 100 comprises a through bore 118 extending from the distal end region 108 to the proximal end region 106 to allow a fluid to flow through the device from the suction tube 112. The through bore 118 is disposed substantially centrally, i.e. axially, through the neck portion 104 and the body portion 102, and tapers gradually outwardly along its length towards the proximal end region 106 of the device. However, optionally, the bore 118 initially tapers inwardly from the open end of the angled distal end region 108 to the start of the neck portion 104 at which point the bore 118 gradually tapers outwardly towards the proximal end region 106 of the device. The optional opposed tapering of the bore in the distal end region 108 and the neck portion 104 allows correspondingly tapered injection moulding pins to be easily removed in opposed directions from the moulded article during manufacture of the device. The slight narrowing of the bore in the distal end region 108 also acts to accelerate and draw fluid from the suction tube 112 and into the neck portion 104 to prevent any build up or blockages in the relatively narrow suction tube and around the angled bend 103 of the neck portion.

The through bore 118 has a minimum diameter of around 2 mm at the junction 103 between the distal end region 108 and the neck portion 104, and a maximum diameter of around 15 mm at the proximal end 106 of the device. The distal end opening is around 3 mm in diameter but may be up to 8 mm for particular applications. The gradual outward tapering of the through bore from the neck portion 104 and through the body portion 102 desirably prevents the build-up of solid material, such as blood or tissue, which could undesirably lead to reduced suction or complete blockage of the through bore. The inner surface of at least the through bore in the neck portion 104 of the device is also substantially continuous along its length with no sudden angles, steps, crevices, or gaps otherwise caused by sudden changes in wall direction and connections/joints which can lead to blockages and/or fluid turbulence and in turn compromised suction and noise. Any undesirable steps along this relatively narrow distal region of the through bore can cause air turbulence resulting in noise and/or refuse, such as blood, being transferred from the surgical site to dry leading to build-up and blocking of the through bore in use.

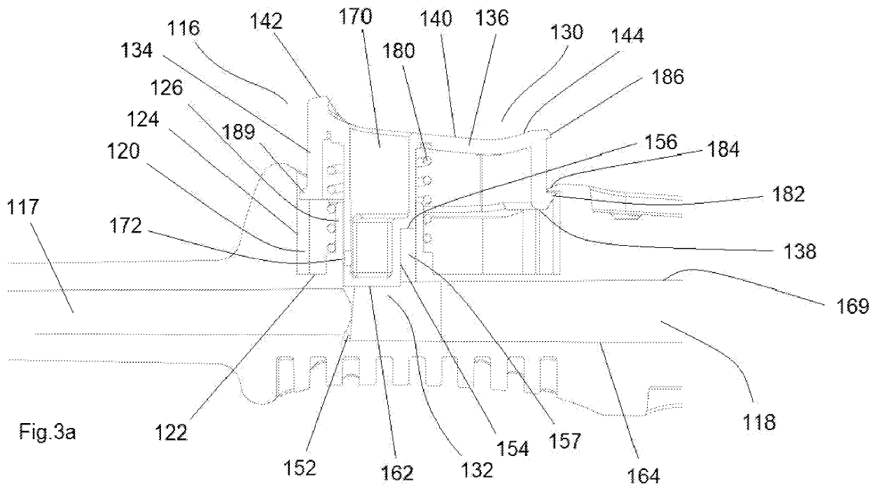

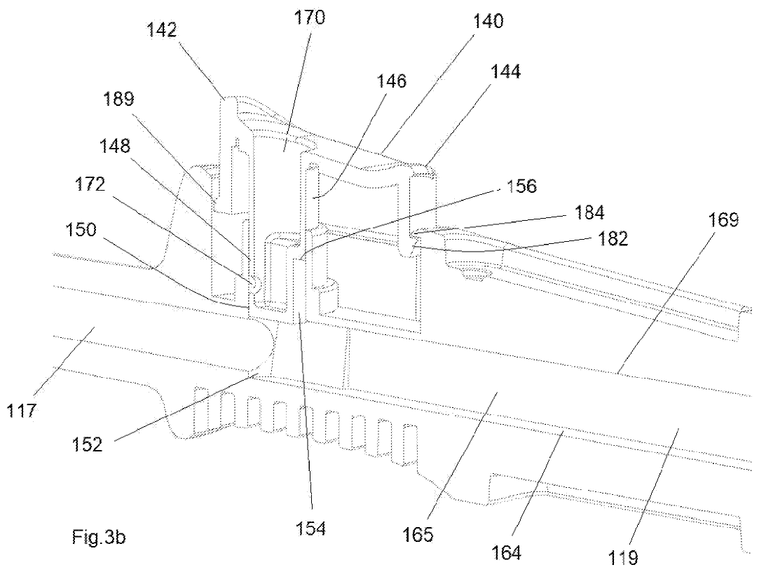

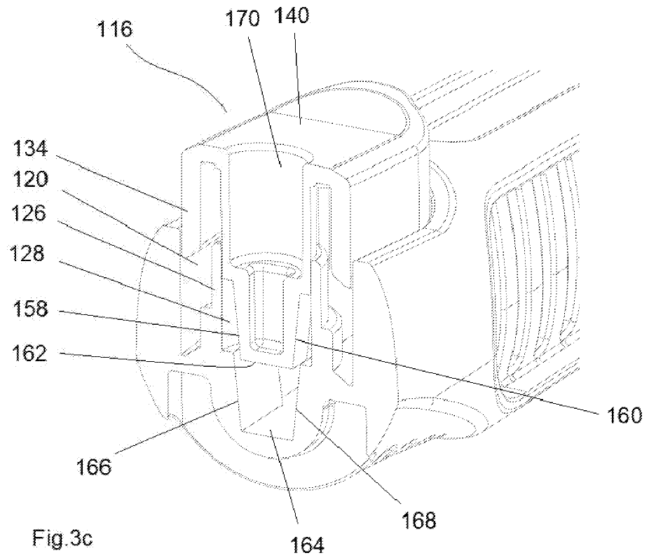

As illustrated in FIGS. 3a to 3c, the body portion 102 includes a cavity 120 extending from its upper outer surface towards the through bore 118. The cavity 120 is defined by a base surface 122 and a continuous side wall surface 124. The cavity opening defines a substantially rounded rectangle in shape. A hole in the base of the cavity 120 communicates with the through bore 118 and is surrounded by a hollow boss 126 extending upwardly from the base surface 122 towards the cavity opening. The cylindrical hollow boss 126 terminates below the opening of the cavity and defines a circular guide bore 128 extending substantially perpendicularly into the through bore 118. The cavity 120 is configured in size and shape to at least partially receive and accommodate the control member 116 which is mounted therein to allow the same to move inwardly and outwardly with respect to the body portion 102 in a direction substantially perpendicular to the axis of the through bore 118.

The control member 116 includes a button portion 130 and an elongate valve portion 132 extending downwardly from an under surface of the button portion. The button portion 130 is substantially elongate in plan view having curved front and rear ends to define a rounded rectangle, but it may be substantially oval, circular, rectangular, or the like. The button portion 130 comprises a continuous side wall/skirt region 134 extending downwardly from an upper region 136 to define a bell or cap-shaped button portion having a substantially open lower edge region 138. The upper region 136 defines an upper surface 140 for locating a user's thumb or finger to selectively urge (push) the button portion 130 of the control member 116 into the cavity 120 and move the valve portion 132 towards a closed position, as described further below. The upper region 136 of the button portion 130 comprises an upturned front edge or front wall 142 and an upturned rear edge or rear wall 144 to accommodate the user's finger or thumb and allow them to efficiently and comfortably operate the control member 116, as described further below.

The valve portion 132 extends downwardly from a distal (forward) region of the button portion 122 and beyond the lower edge region of the skirt 134. The button portion 130 and the valve portion 132 define a mushroom-shape and are integrally formed, but they could be separate components connected together. An integrally formed control member 116 desirably reduces the number of moving/coupled parts, reduces assembly time and cost, and provides a non-complex control mechanism wherein the control member acts as a button and a valve element.

The valve portion 132 is substantially elongate and an upper region 146 thereof has a substantially circular cross section having an outer diameter which corresponds with an inner diameter of the circular guide bore 128 such that at least the upper region 146 of the valve portion 132 slidably engages with and is guided by the guide bore 128 when the control member 116 is selectively moved between valve open and closed positions by the user. The interface between the valve portion 132 and the guide bore 128 need not be a sealed interface. The upper region 146 of the valve portion 132 and the guide bore 128 are circular in cross section but may be substantially elliptical, hexagonal, square or the like in cross section. A lower region 148 of the valve portion 132 has a curved front/distal surface 150 corresponding with the curvature of a front side of the upper region 146 of the valve portion 132 to define a continuous convex surface of uniform curvature extending along the length of the front side of the valve portion. The front curved surface 150 of the lower region of the valve portion slidably engages with a correspondingly curved surface 152 of the through bore 118 which is a continuation of the guide bore 128 and defined at an intersection of a distal bore region 117 and a proximal bore region 119 of the through bore 118.

The distal bore region 117 which extends through the neck portion 104 of the device 100 is substantially circular in cross section, but it may alternatively be elliptical or oval or the like. The proximal bore region 119 has four substantially flat inner surfaces extending along its length, but it may alternatively be elliptical or oval or the like along at least some of its length aptly downstream of the valve portion 132 and the valve seat surfaces of the proximal bore region 119. The proximal bore region 119 is at least partially defined by a flat base surface 164, opposed flat side surfaces 165 and a flat upper surface 169. The base surface 164 of the proximal bore region 119 is lower than the distal bore region 117 such that the curved surface 152 of the through bore 118 extends downwardly beyond the distal bore region 117 to meet the base surface 164 of the proximal bore region 119.

As best illustrated in FIGS. 3a and 3b, a rear surface 154 of the lower region 148 of the valve portion 132 is substantially flat to define an undercut/shoulder surface 156. The flat rear surface 154 and shoulder surface 156 extend laterally with respect a longitudinal axis of the device and define a gap 157 between the valve portion 132 and the guide bore 128. The gap 157 desirably reduces the slidable contact between the valve portion 132 and the guide bore 128 to thereby reduce stiction when the control member 116 is in the open position.

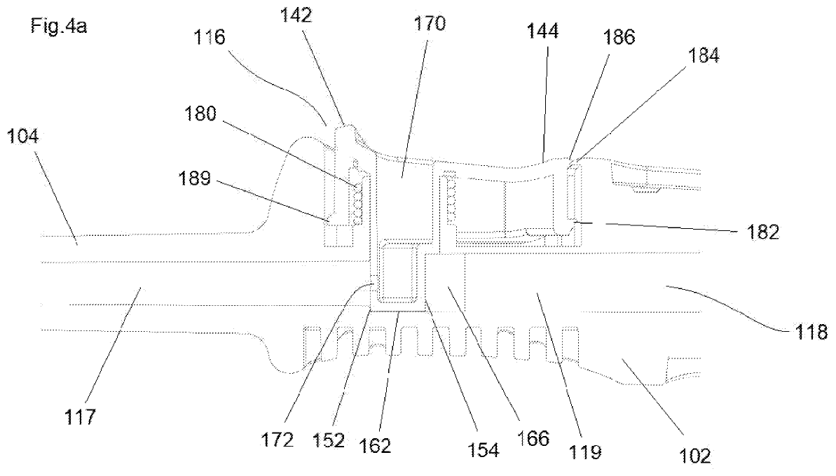

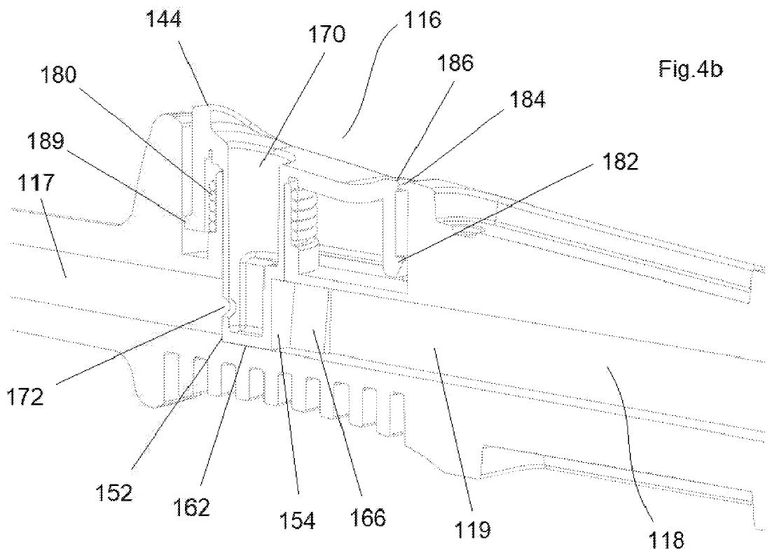

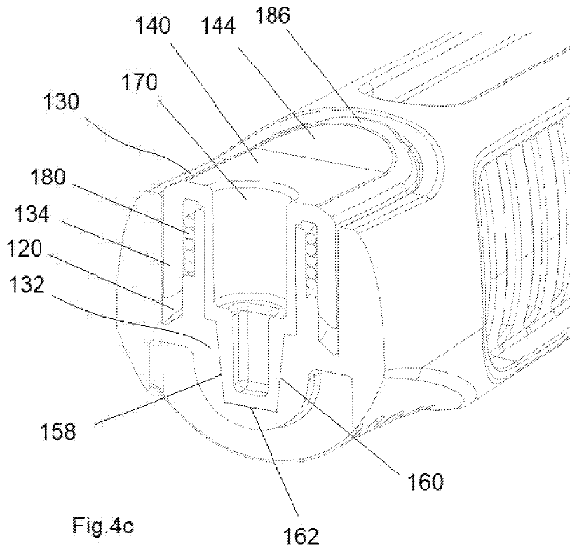

As illustrated in FIG. 3c, opposed side surfaces 158,160 of the lower region 148 of the valve portion 132 are substantially flat and taper outwardly from a flat base surface 162 of the valve portion 132 in an upward direction towards the button portion 130. When the control member 116 is in the closed position, as illustrated in FIGS. 4a to 4c, the flat base surface 162 of the valve portion 132 engages with the flat base surface 164 of the through bore 118, and the flat and tapered side surfaces 158,160 of the valve portion 132 engage respectively with flat and correspondingly tapered side surfaces 166,168 of the through bore 118. The tapered surfaces provide an efficient seal when urged together, whilst preventing stiction to allow the control member 116 to return to the open position when thumb/finger pressure is released from the button portion 130 of the control member 116. The tapered surfaces are aptly around 6 degrees relative to the vertical axis. The curved front surface 152, the flat base surface 164 and the tapered side surfaces 166, 168 of the proximal bore region 119 act as valve seat surfaces against which the corresponding surfaces of the lower region 148 of the valve portion 132 engage when the control member 116 is in the closed position.

The opposed tapered side surfaces 166, 168 of the through bore which act as valve seat surfaces may optionally include at least one groove, such as a plurality of spaced apart and parallel grooves, extending up each tapered surface to reduce the contact area between the valve portion 132 and the tapered side seat surfaces, and in turn any stiction effects, when the valve portion of the control member 116 is in the closed or locked positions, as described further below, whilst still providing an efficient seal between the correspondingly tapered surfaces when they are urged together.

The control member 116 also includes a vent bore 170 extending downwardly from the upper surface 140 of the button portion 130 and along the valve portion 132 towards the closed base of the valve portion. The vent bore 170 is oriented substantially perpendicularly with respect to the through bore 118 of the device. A vent hole 172 is disposed in the curved front surface 150 of the lower region 148 of the valve portion 132 and proximally above the base of the vent bore 170 to communicate therewith. As illustrated in FIGS. 3a and 3b, the vent hole 172 is closed off by the guide bore 128 when the control member 116 is in the open position and, as illustrated in FIGS. 4a and 4b, the vent hole 172 is located in fluid communication with the distal bore region 117 when the control member 116 is in the closed position.

The control member 116 is mounted on a compression spring 180 which biases the valve portion 132 towards the open position (as illustrated in FIG. 3a). The spring 180 is mounted on the guide boss 126 and engages with the under surface of the button portion 130 of the control member 116. The spring, or other suitable biasing element, may engage a different region of the control member 116 to urge the valve member towards the open position when no force is being applied to the button portion 130 by a user. For example, the biasing element/s may include a flexible yet resilient plastic lever, a compressible rubber or foam element, or the like. The spring may be a linear or progressive spring.

The control member 116 may alternatively be configured such that it is urged towards a closed position by a biasing element, such as a compression or tension spring, and the user must move the control member, and in turn the valve portion, towards the open position to selectively control a suction at the distal end of the device.

The control member 116 may alternatively be configured to slide longitudinally or rotate with respect to the body portion 102 of the device to selectively control the flow of air through the device, and in turn a suction pressure at the distal end. For example, the button portion may be a slider moveable by the user's finger or thumb along a track which in turn moves the valve portion with respect to the through bore to adjust the flow of fluid through the device. Alternatively, the button portion may be a knob rotatable by the user to move the valve portion with respect to the through bore to adjust the flow of fluid through the device.

As illustrated in FIGS. 3a and 3b, the control member 116 further comprises at least one lower projection 182, such as a flange or shoulder, extending outwardly from the rear/proximal lower end region of the skirt 134 of the button portion 132 which is configured to engage with a corresponding lip region 184 extending inwardly from an upper rear edge region of the cavity 120 of the body portion 102. The lip region 184 acts as a stop and limits the travel of the control member 116 urged by the spring 180 to thereby couple the control member 116 to the body portion 102 when the control member 116 is in the open position corresponding to a fully-open through bore 118. The lower projection 182 may be a plurality of spaced apart projections extending around at least a rear/proximal end region of the skirt 134 or it may comprise a continuous flange or shoulder extending around at least the rear/proximal end region of the skirt 134. Aptly, the lower flange 182 may extend around the periphery of the skirt 134 and the corresponding lip region 184 may extend around the periphery of the aperture 120.

The control member 116 further includes at least one upper projection 186 extending outwardly from the rear/proximal upper end region of the button portion 130 which is configured to engage with the lip region 184 extending inwardly/forwardly from the upper rear edge region of the aperture 120 of the body portion 102. The upper projection 186 is aptly a flange region extending outwardly from the button portion 130 to continue the upper surface 140 of the control member 116. When the control member 116 is depressed into the cavity 120 such that the upper flange 186 engages with the lip region 184, the upper flange acts as a stop to limit the movement of the control member into the cavity of the device. In this position, the control member 116 is in the closed position and the valve portion 132 thereof is located across the through bore 118. Alternatively, the device may be configured such that the valve portion is urged towards the closed position when no force is exerted on the control member and the valve portion may be locked in the open position. Further alternatively, the valve member may be locked in one or both of the fully open and fully closed positions and/or one of a number of different positions between the fully open and fully closed positions, such as 25%, 50% etc.

Further depression of the control member 116 into the cavity 120 urges the upper flange 186 past and under the lip region 184 such that the lip region constrains the control member against the reaction force of the spring 180 and prevents it returning to the open position when a user's digit is removed. The control member 116 is now in a locked position and no fluid can flow through the device from the distal bore region 117 to the proximal bore region 119, as described further below. Allowing the button portion 130 of the control member 116 to slightly tilt or rotate in a forward-aft direction within the cavity 120, in view of for example a slight flex/resilience of the elongate valve portion 132, allows the upper flange 186 to clear the lip region 184 to lock/unlock the control member 116 as desired. Alternatively, or additionally, the upper flange 186 and/or lip region 184 may be substantially resilient and flex slightly to allow the upper flange 186 to be forced past the lip region 184 to thereby move the control member 116 from the closed position to the locked position, or vice versa. Further alternatively, or additionally, the upper flange 186 and/or lip region 184 may be substantially curved, tapered, bevelled or the like to allow the upper flange 186 to efficiently pass the lip region when being urged by the user to or from the locked position. A lower flange 189 extends from the lower front edge of the skirt region 134 of the button portion 130 for engagement with the front inner surface of the cavity 120 and to act as a pivot when the button portion 130 is tilted/rotated forwardly to lock and/or unlock the control member 116. Alternatively, a locking element may be movable by the user to engage with a portion of the control member, such as the button portion, to thereby lock it in the locked position. For example, a locking element may be slidably movable with respect to the body portion to at least partially cover the button portion or engage therewith and prevent the control member being urged towards the open position by the spring.

Full suction of around −60 to −80 dPa is achieved at the distal tip of the device when the valve portion 132 is in the open position, i.e. when no force is being applied to the control member 116 and the spring 180 has urged the same into the open position, as illustrated in FIGS. 3a to 3c.

A relatively light/soft suction is achieved at the tip of the device when the control member 116 is pressed by the user into the closed position and with their finger/thumb covering the vent hole in the top of the button portion 132. Fluid flow is significantly reduced when the valve portion 130 is in the closed position and the negative pressure upstream of the valve is reduced to around −40dPa. The negative pressure at the tip of the device gradually increases to around −60 to −80 dPa as the pressure differential across the valve portion equalises. Desirably, yet optionally, a perfect/complete seal is not created/achieved when the control member 116, and in turn the valve portion 132 thereof, is in the closed position as a result of design tolerances and surface roughness, i.e. fluid (air) flows past the valve portion via micro-passageways. Alternatively, or additionally, at least one more defined passageway, such as a groove, may be disposed along the flat base surface of the valve portion 132 opening at the front and rear surfaces thereof such that fluid flow is allowed to leak through the passageway when the valve portion is in the closed position but the front opening of the passageway is closed off when the valve portion is urged forwardly against the corresponding curved front seat surface when the control member 116 is in a locked position, as described further below. The increased negative pressure in the proximal bore region 119, and in turn the pressure differential across the valve portion 132, also acts on the rear face of the valve portion 132 to urge the front curved surface 150 of the valve portion 132 away from the front curved surface 152 of the through bore 118 allowing fluid (air), albeit at a relatively low/minimal rate, to leak past the valve portion from the distal bore region to the proximal bore region and thereby create a relatively low/soft suction at the tip of the device to draw fluid and/or solids, such as blood, bone chips or earwax, into the suction tube 112 and possibly into the distal bore region 117 of the device at a relatively low volume. In view of the outwardly tapered inner surfaces of the suction tube 112, earwax for example is drawn into the suction tube without the same becoming blocked and the ‘soft’ suction pressure remains substantially constant. If the inner surfaces of the suction tube were substantially parallel, as with many conventional suction tubes, the same would become blocked due to the frictional effects between the inner surfaces and the earwax being drawn further into the suction tube. The outward tapering of the distal bore region 117 towards the valve portion 132 also allows earwax or other solids to be collected in the distal bore region 117 without blocking. This slow leak function when the control member/button 116 is held down in the closed position by a user's finger or thumb allows earwax, for example, to be gently removed at particularly low noise levels and reduces/eliminates any risk of acoustic shock or other discomfort to the patient.

A user's finger or thumb can also be placed over the vent bore 170 when the control member 116 is in the closed position to gradually increase the relatively low negative pressure in the distal bore region 117 and in the suction tube 112 for ‘soft suction’ applications, e.g. gently extracting ear wax or picking up, manoeuvring and locating fine and lightweight prostheses, such as ear grommets, with the device during a surgical procedure. Removal of the user's thumb from the vent bore 170 fluidly communicates the distal bore region 117 of the device to atmosphere via the vent hole 172 which is located in the through bore 118 when the control member 116 is in the closed position. This allows air to enter the distal bore region 117 via the vent bore 170 and the vent hole 172 which in turn releases the negative pressure therein and the low/soft suction at the tip of the suction tube to thereby release the prosthesis from the device and implant the same in the desired location.

As described above, the control member 116 can be placed in a locked down position wherein the rear/proximal region of the button portion 130 is pressed further downwardly from the closed position to thereby urge the upper flange 186 past and under the lip region 184 of the cavity 120. An upper surface of the lip region is angled downwardly to help guide the upper flange 186 over and under the lip region 184. Whilst the control member 116 is formed from a relatively stiff material, such as Polyoxymethylene (POM), it does have a degree of resilience to allow the rear region of the button portion 130 to flex slightly and pivot around the rear upper edge of the guide bore 128 when being pushed downwardly and under the lip region 184 of the surrounding cavity 120. Aptly, POM has a particularly low coefficient of friction which ensures the control member 116 efficiently slides within the guide bore 128 between open and closed positions. In view of the resilience of the material, albeit relatively low, a rotational reaction force is transferred through the control member 116 which urges the lower end of the valve member 132 forwardly which in turn urges the lower curved surface of the valve member against the curved seal surface 152 of the proximal bore region 119. Aptly, the flat rear surface 154 and flat base surface 162 of the valve portion 132 define a straight lower rear edge of the valve portion which ensures an efficient seal entirely along that lower edge is maintained with the flat base of the bore 119 when the valve portion tilts backwardly slightly on that lower edge, which would not be the case if the rear face of the valve member, and in turn the lower rear edge, were curved. The downward force on the control member also urges the base surface and tapered surfaces of the valve portion 132 further against the base surface and correspondingly tapered seal surfaces of the proximal bore region 119. As such, a substantially complete seal is achieved to block off the distal bore region 117 from the proximal bore region 119 when the control member 116 is in the locked down position. In view of the sliding interface between the valve portion 132 and the guide bore 128, air may be drawn into the proximal bore region 119 via the guide bore, however suction at the distal tip of the device will be zero or at least negligible to allow the ‘locked down’ position of the control member 116 to be used as an ‘emergency stop’ function if tissue is being aggressively held at the tip and/or if the patient is experiencing discomfort.

To unlock the control member 116 from the locked down position, the button portion 130 is urged forwardly by the user's thumb or finger to thereby slightly rotate/tilt the same in the cavity. The lower flange 189 extending from the lower front edge of the skirt region 134 of the button portion 130 may act as a stop to limit the forward movement of the button portion 130 in the cavity 120 but also acts as a pivot when rotating the button portion forwardly. The forward movement and slight forward rotation urges the upper flange 186 past the lip region 184 of the cavity 120 and in turn allows the control member to be urged back towards the open position by the spring 180. The upturned front and rear upper edges of the button portion 130 desirably contain the user's thumb thereon and provide a surface to push against when the button portion 130 is being selectively tilted/rotated during the locking or unlocking operations.

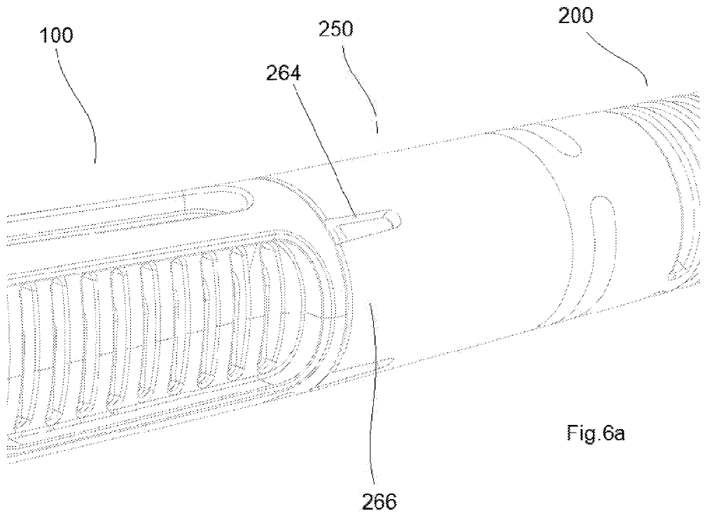

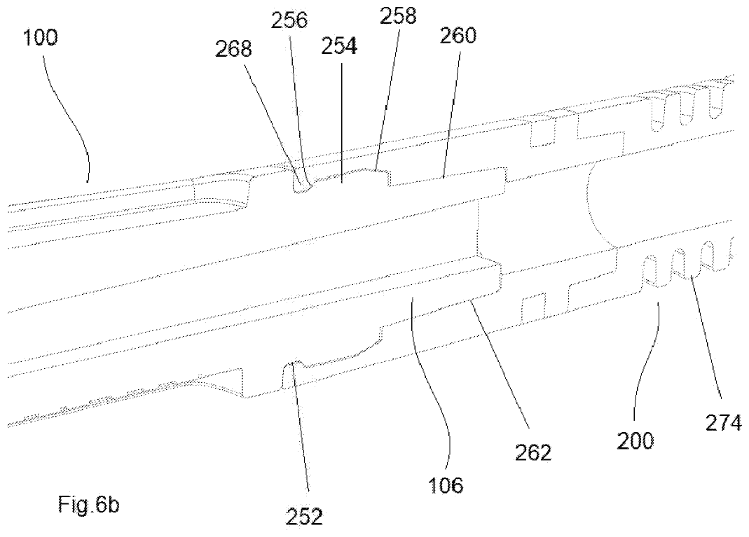

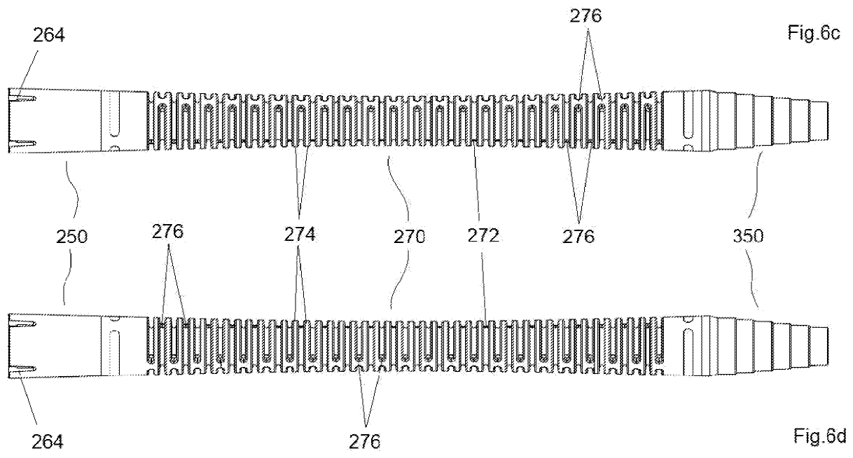

As illustrated in FIGS. 6a and 6b, the proximal end region 106 of the device 100 is configured to connect to an end fitting 250 of a flexible tube 200 coupled to a source of negative pressure, such as a vacuum pump. The proximal end region 106 of the device comprises an annular recess 252 adjacent to an enlarged shoulder portion 254 having tapered distal and proximal end surfaces 256,258. The proximal end region 106 of the device tapers outwardly from a proximal end of the device to the enlarged portion 254 to provide a tapered outer engagement surface 260.

The end fitting 250 of the flexible tube 200 comprises an inner engagement surface 262 which tapers inwardly from the open end region of the end fitting 250. The angle of taper of the inner engagement surface 262 of the end fitting corresponds with the angle of taper of the outer engagement surface 260 of the proximal end region of the device to thereby provide a sealed connection therebetween when the end fitting 250 is connected to the device. The end fitting 250 includes a plurality of circumferentially spaced slots 264 extending in a longitudinal direction from the opening of the end fitting. These slots define spaced apart resilient end portions 266 which flex radially outwardly when the end fitting is axially urged over the proximal end region of the device. The end portions 266 of the end fitting each include a projection 268, such as a part-annular rib, which snap into the annular recess 252 of the proximal end region 106 of the device to securely connect the end fitting to the device in a sealed manner. Alternatively, the end fitting 250 may be a sufficiently flexible yet resilient material to expand outwardly when urged over the proximal end region 106 of the device to thereby not require the spaced apart slots 264 defining the resilient end portions 266.

The tapered proximal end surface 258 of the shoulder portion 254 acts to guide the end portions 266 of the end fitting over the shoulder portion 254 and urge the same radially outwardly until they pass over the shoulder portion 254 and snap into the annular recess 252. The tapered distal end surface 256 of the shoulder portion 254 cooperates with a correspondingly tapered surface of each rib 268 to thereby urge the end fitting 250 axially onto the device as the end portions 266 resiliently want to enter the annular recess 252. These interfaces provide a substantially efficient seal between the end fitting and the device.

The tapered outer surface 260 also engages with a corresponding surface inside the end fitting to provide a further sealing interface. Desirably the circular cross section of the proximal end region 106 of the device and of the correspondingly sized and shaped end fitting 250, and in particular the snap-fit relationship between the annular recess 252 and the end fitting rib/s 268, allows the end fitting, and in turn the flexible tube 200, to rotate in use with respect to the device, and vice versa. This eliminates the risk of the flexible tube becoming twisted and/or exerting a reaction torque on the device during a surgical procedure. This arrangement also allows for quick connection and disconnection of the device to and from the flexible tube, and also provides a continuous and smooth transition from the device to the tube to eliminate any turbulence effects, such as noise, and to prevent blocking at the connection interface/s.

As illustrated in FIGS. 6b to 6d, the flexible tube 200 includes an end fitting 250,350 at each end of a substantially flexible tube member 270. The tube member 270 comprises a tubular wall portion 272 providing a continuous inner tube surface for transporting a fluid, and a plurality of longitudinally spaced apart annular portions 274 arranged substantially parallel to each other. The wall portion 272 and the annular portions 274 are integrally formed by, for example, injection moulding or the like. Alternatively, the annular portions 274 may be part of a separate structure through which the wall portion 272 is located. For example, the tubular wall portion 272 may be a flexible tube located inside a structure comprising the annular portions 274 which protect the wall portion 272.

Adjacent ones of the annular portions 274 are coupled together by a longitudinally oriented hinge element 276. The hinge elements 276 are arranged along radially opposed sides of the flexible tube and are staggered such that a first hinge element is located on a first side of the tube, the second hinge element is located on the second side of the tube opposite the first side, the third hinge element is arranged on the first side of the tube, and so on. This arrangement allows the tube to flex in all four directions, i.e. up, down, left and right. The annular portions 274 are also relatively rigid compared to the relatively flexible tubular wall portion 272 and protect the tubular wall portion. The rigid annular portions 274 also prevent collapse and blocking of the tubular wall portion 272 when the same is subject to a negative pressure by the vacuum source. Aptly, the tube member 270 is made from a plastics material such as a thermoplastic elastomer and the end fittings 250,350 are made from a polycarbonate.

Certain embodiments of the present invention therefore provide a suction device for accurately and efficiently removing fluids from a surgical site, such as during an ENT procedure or to remove fluid from the upper airway whilst a patient is under an anaesthetic. The suction device is comfortable to use for relatively long periods of time, and allows a user to accurately and efficiently control the amount of suction applied by the device to a surgical site, such as in the ear, nose, throat or intracranial cavity. The suction device is easy and quick to connect to a vacuum source, such as a pump, and does not cause the flexible tubing coupling the device to the vacuum source to become twisted in use. The suction device can be used with a selected one of a plurality of differently configured suction tubes which are inexpensive and non-complex to manufacture, sterilise or recycle. The suction device is less prone to blocking and is quieter than conventional suction devices, particularly when suction is not required. The suction device according to certain embodiments of the present invention is configured to allow a user to select between a full suction mode, a soft suction mode, and a variety of different suction pressures therebetween, and also to substantially shut-off the suction at the tip of the device if desired. The suction device is also configured to allow a user to gently extract wax from an ear without the risk of acoustic injury or other discomfort to the patient. The suction device is also configured to allow a user to efficiently pick up, manoeuvre, locate and release a prosthesis in a desired implant location, such as an ear grommet within the ear of a patient.

Whilst the device according to certain embodiments of the present invention is particularly suitable as a suction device for the medical industry, the device may be suitable for non-medical applications and/or may be used as a blower device for blowing fluids or solids, such as compressed air or the like, at an object or as a dispensing device for dispensing fluids or solids at a target location. The tapered inner bore is suitable for compressing a fluid being forced through the device from the proximal bore region towards the distal end of the device and in turn increasing a pressure and flow rate of the fluid, such as air, leaving the distal end of the device. The control member is aptly configured to allow a user to accurately control the pressure and/or velocity of a fluid exiting the nozzle. The tapered nozzle is also aptly configured to accurately localise compressed air or the like at an object.

1-25. (canceled)

26. A device for transferring a fluid from a surgical site, comprising:
an elongate body portion defining a proximal end region of the device connectable to a source of negative pressure;
an elongate neck portion defining a distal end region of the device locatable at a surgical site;
a through bore extending from the distal end region to the proximal end region; and
a control member for selectively controlling a negative pressure at the distal end region of the device,
wherein:
the control member is slidably mounted in an aperture of the body portion to allow the same to move inwardly and outwardly with respect to the body portion and comprises a valve portion extending downwardly from a button portion actuatable by a user to selectively move the valve portion across the through bore from an open position towards a closed position to substantially restrict a fluid flowing along the through bore from the distal end region and past the valve portion to thereby substantially reduce but not stop the negative pressure at the distal end region of the device, and
the valve portion is moveable from the closed position to a sealed position wherein at least one seal surface of the valve portion is urged against a respective seat surface of the through bore to thereby prevent fluid flow past the valve portion.

27. The device according to claim 26, wherein the control member comprises a vent bore extending from an upper surface of the button portion towards a closed base of the valve portion, and a vent hole disposed in a distal side of the valve portion such that the vent hole is closed off when the control member is in the open position and the vent hole is located in fluid communication with a distal bore region of the through bore when the control member is in the closed position.

28. The device according to claim 26, wherein a lower region of the valve portion comprises a curved front surface for engaging a correspondingly curved seat surface of the through bore which is a continuation of the guide bore and defined at an intersection of a distal bore region and a proximal bore region of the through bore.

29. The device according to claim 28, wherein the lower region of the valve portion comprises a substantially flat base surface for engagement with a correspondingly flat base seat surface of the proximal bore region when the valve portion is in the closed position, and wherein the lower region of the valve portion further comprises substantially tapered and flat side surfaces for engagement with correspondingly tapered and flat side seat surfaces of the proximal bore region when the valve portion is in the closed position.

30. The device according to claim 29, wherein the lower region of the valve portion comprises a substantially flat rear surface which meets the flat base surface to define a straight lower rear edge of the valve portion.

31. The device according to claim 26, wherein the valve portion of the control member is biased towards the open position by a biasing element, and the button portion of the control member comprises a first abutment surface for engagement with a first stop surface of the body portion of the device when the valve portion is in the open position and to limit travel thereof.

32. The device according to claim 31, wherein the first abutment surface is provided by a first flange region extending proximally from a lower region of the button portion, and the first stop surface is provided by a lip region extending distally from an upper edge region of the cavity.

33. The device according to claim 31, wherein the button portion comprises a second abutment surface for engagement with a second stop surface of the body portion when the valve portion is in the closed position and to limit travel thereof.

34. The device according to claim 33, wherein the second abutment surface is provided by a second flange region extending proximally from an upper region of the button portion, and the second stop surface is provided by the lip region.

35. The device according to claim 33, wherein the button portion comprises a third abutment surface for engagement with the second stop surface to hold the valve portion in the sealed position.

36. The device according to claim 35, wherein the third abutment surface is provided by the second flange region and the second stop surface is provided by the lip region.

37. The device according to claim 36, wherein the second flange region and/or the lip region is substantially resilient to allow the second flange region to be urged past the lip region and engage thereunder when the control member is moved from the closed position to the sealed position.

38. The device according to claim 36, wherein the button portion is tiltable towards the proximal end region of the device to locate the second flange region under the lip region and/or is tiltable towards the distal end region of the device to release the second flange region from under the lip region.

39. The device according to claim 26, wherein an inner surface of the through bore tapers outwardly in a distal to proximal direction.

40. A system for transferring a fluid from a surgical site, comprising:
the device according to claim 26;
at least one suction tube mountable on the distal end region of the device; and
optionally further comprising a flexible tube connectable to the proximal end region and rotatable with respect to the device.

41. Use of a device according to claim 26 for transferring a fluid or solid from a target site.

42. A method of selectively controlling a negative pressure at a distal end region of a suction device, the method comprising:
actuating a button portion of a control member of the suction device to move a valve portion of the control member across a through bore of the device from an open position to a closed position to substantially restrict a fluid flowing along the through bore from the distal end region and past the valve portion to thereby substantially reduce but not stop the negative pressure at the distal end region of the device.

43. The method according to claim 42, comprising picking up an object at the distal end region of the device when the valve portion is in the closed position.

44. The method according to claim 42, comprising moving the control member from the closed position to a sealed position wherein at least one seal surface of the valve portion is urged against a respective seat surface of the through bore to thereby prevent fluid flow past the valve portion.

45. The method according to claim 44, comprising at least partially removing a user's finger or thumb from the button portion to at least partially uncover an opening in the button portion to allow a negative pressure in a distal bore region of the through bore and upstream of the valve portion to release to atmosphere via a vent of the control member in fluid communication with the distal bore region when the valve portion is in the sealed position.
